(19) United States
(12) Patent Application Publication
(10) Pub. No.: US 2025/0302288 A1
(43) Pub. Date: Oct. 2, 2025

(54) OPTICAL DETECTION APPARATUS, ENDOSCOPIC TIP DEVICE AND BIOMEDICAL MONITORING DEVICE

(71) Applicant: Yuan LU, Nancy (FR)

(72) Inventor: Yuan LU, Nancy (FR)

(21) Appl. No.: 19/074,138

(22) Filed: Mar. 7, 2025

Related U.S. Application Data
(60) Provisional application No. 63/570,297, filed on Mar. 27, 2024.

(51) Int. Cl. A61B 1/06

(52) U.S. Cl. CPC A61B 1/0676

(57) ABSTRACT

Disclosed is an optical detection apparatus. At least one spin-based light emitting device emits a circularly polarized light towards an object to be detected. A plurality of spin-based photodiodes are arranged around the spin-based light emitting devices, and are configured to detect the polarization state of light scattered back from the object. The spin-based light emitting device is a surface-emitting device having a first surface for emitting light. The spin-based photodiodes are surface-illuminated photodiodes each having a second surface for receiving the light scattered back from the object. The circular polarization of the light emitted from the spin-based light emitting device can be modulated through switching the magnetization direction. In some embodiments, the magnetization direction of the spin injector in the spin-based light emitting device and/or the spin detector in the spin-based photodiode can be switched by applying pulsed current.

### CROSS-REFERENCE TO RELATED APPLICATIONS

This application claims the benefit of U.S. Provisional Application No. 63/570,297, filed on Mar. 27, 2024, the disclosure of which is incorporated herein by reference in its entirety and for all purposes.

### TECHNICAL FIELD

The disclosure herein relates to an optical detection apparatus for biomedical applications.

### BACKGROUND

Studies indicate that the polarization state of light scattered by biological tissues can reveal their structural details, making polarized light scattering a valuable tool for differentiating structurally similar biological systems and monitoring temporal structural changes. For example, the enlargement of cell nuclei during carcinogenesis alters the polarization state of the scattered light. Leveraging this principle, polarimetry using circularly polarized light (CPL) or linearly polarized light (LPL) emerges as a non-invasive diagnostic technique, offering critical insights for the early disease detection and enhancing the diagnostic capabilities of pathologists.

### SUMMARY

According to an aspect of the disclosure, an optical detection apparatus is provided, comprising: at least one spin-based light emitting device configured to emit a circularly polarized light towards an object to be detected, the spin-based light emitting device is a surface-emitting device having a first surface for emitting light; and a plurality of spin-based photodiodes arranged around the at least one spin-based light emitting device and configured to detect the polarization state of light scattered back from the object, the spin-based photodiodes are surface-illuminated photodiodes each having a second surface for receiving the light scattered back from the object.

Optionally, the second surface is either coplanar with or parallel to the first surface.

Optionally, a central normal line of the second surface is oriented toward the region of the object illuminated by the light from at least one spin-based light-emitting device.

Optionally, the spin-based light emitting device comprises: a first multi-layer semiconductor structure comprising gain medium of quantum dots or quantum wells, the gain medium of quantum dots or quantum wells are capable of emitting light with circular polarization state determined by the spin direction of the injected spin-polarized carriers; and a spin injector configured to inject spin-polarized carriers into the first multi-layer semiconductor structure.

Optionally, the spin injector is in a form of a bar-shaped channel, the spin-based light emitting device further comprises a first electrode and a second electrode respectively connected to two opposite ends of the bar-shaped channel to apply a pulsed current into the bar-shaped channel, so as to switch the magnetization direction of the spin injector, wherein the spin direction of the spin-polarized carriers injected from the spin injector into the first multi-layer semiconductor structure is determined by the polarized magnetization direction of the spin injector.

Optionally, alternating reverse pulsed current is applied into the bar-shaped channel to alternatively reverse the magnetization direction of the spin injector.

Optionally, the spin-based light emitting device further comprises: a first substrate, wherein the first multi-layer semiconductor structure is sandwiched between the first substrate and the spin injector; a third electrode connected to the spin injector; and a fourth electrode connected to the first substrate, wherein the third electrode and the fourth electrode are configured to apply a first voltage between the spin injector and the first substrate to inject carriers from the spin injector into the first multi-layer semiconductor structure.

Optionally, the spin-based light emitting device further comprises: a bottom distributed Bragg reflector, wherein the first multi-layer semiconductor structure is sandwiched between the spin injector and the bottom distributed Bragg reflector.

Optionally, the spin-based light emitting device further comprises: a top distributed Bragg reflector, wherein the spin injector is sandwiched between the first multi-layer semiconductor structure and the top distributed Bragg reflector, and an intracavity resonant surface emitting laser structure is formed between the top distributed Bragg reflector and the bottom distributed Bragg reflector.

Optionally, a surface area of the spin injector is large enough to cover the first multi-layer semiconductor structure to ensure a homogenous carrier injection into the gain medium.

Optionally, the distance between the spin injector and the gain medium of quantum dots or quantum wells is configured to place the spin injector in one node of the stationary electromagnetic field formed by the light reflected from the top and bottom distributed Bragg reflectors.

Optionally, the spin-based photodiodes comprises: a second substrate; a second multi-layer semiconductor structure formed above the second substrate, the second multi-layer semiconductor structure is capable of creating spin-polarized carriers with the illumination of circularly polarized light; and a spin detector formed above the second multi-layer semiconductor structure, and the spin detector is capable of detecting a helicity dependent spin photocurrent flow through the spin detector.

Optionally, the spin-based photodiodes further comprises: a fifth electrode connected to the spin detector; a sixth electrode connected to the second substrate, wherein the fifth electrode and the sixth electrode are configured to apply a second voltage between the spin detector and the second substrate to drive the spin-polarized carriers created in the second multi-layer semiconductor structure to the spin detector; and a current meter connected to the fifth electrode and the sixth electrode and configured to detect the helicity dependent spin photocurrent flow through the spin detector.

Optionally, the second multi-layer semiconductor structure comprises a gain medium of quantum dots or quantum wells capable of creating spin-polarized carriers with the illumination of circularly polarized light. And optionally, a bandgap of the gain medium of the spin-based photodiode is smaller than a bandgap of a gain medium of the spin-based light emitting device.

Optionally, the second multi-layer semiconductor structure comprises a PN junction structure capable of creating spin-polarized carriers with the illumination of circularly polarized light.

Optionally, a tunneling barrier in the spin detector of the spin-based photodiode is thinner that a tunneling barrier in the spin injector of the spin-based light emitting device.

Optionally, the spin detector has the same two-dimensional shape as the second multi-layer semiconductor structure when observed from the second surface.

Optionally, the spin detector is in a form of a bar-shaped channel, the vertical-type spin-based photodiode further comprising a seventh electrode and an eighth electrode respectively connected to two opposite ends of the bar-shaped channel of the spin detector to apply a pulsed current into the bar-shaped channel of the spin detector, so as to switch the magnetization direction of the spin detector.

Optionally, the optical detection apparatus further comprising: a processor configured to determine the circular polarization rate of the light beams received by the plurality of spin-based photodiodes.

According to the second aspect of the disclosure, an endoscopic tip device is provided. The endoscopic tip device comprises an optical detection apparatus of the disclosure.

According to the third aspect of the disclosure, a biomedical monitoring device configured to be embedded inside human or animal body for real-time observation on a specific area. The biomedical monitoring device comprises an optical detection apparatus of the disclosure.

According to the fourth aspect of the disclosure, a vertical-type spin-based photodiode with surface illuminated geometry is provided, comprising: a substrate; a multi-layer semiconductor structure formed above the substrate, the multi-layer semiconductor structure is capable of creating spin-polarized carriers with the illumination of circularly polarized light; a spin detector formed above the multi-layer semiconductor structure, and the spin detector is capable of detecting a helicity dependent spin photocurrent; a fifth electrode and a sixth electrode respectively connected to the spin detector and the substrate, wherein the fifth electrode and the sixth electrode are configured to apply a voltage between the spin detector and the substrate to drive the spin-polarized carriers created in the second multi-layer semiconductor structure to the spin detector; and a current meter connected to the two electrodes and configured to detect the helicity dependent spin photocurrent flow through the spin detector.

Optionally, the spin detector is in a form of a bar-shaped channel, the vertical-type spin-based photodiode further comprising a seventh electrode and an eighth electrode respectively connected to two opposite ends of the bar-shaped channel of the spin detector to apply a pulsed current into the bar-shaped channel of the spin detector, so as to switch the magnetization direction of the spin detector.

### BRIEF DESCRIPTION OF FIGURES

By more detailed description of the exemplary embodiments of the present disclosure in combination with the accompanying drawings, the above and other purposes, features and advantages of the present disclosure will become more apparent. In the exemplary embodiments of the present disclosure, the same reference numeral generally represents the same component.

FIG. 1 is a cross-sectional view of the optical detection apparatus according to an embodiment of the disclosure.

FIG. 2 is a schematic view of the arrangement of spin light emitting devices and spin photodiodes in the detection side of the optical detection apparatus according to the embodiment of the disclosure.

FIGS. 3A to 3C show variant arrangements of the light emitting portion and the light detection portion.

FIG. 4A is a schematic view showing relative positional relationships of the components of the light emitting device according to an embodiment of the disclosure.

FIG. 4B is a schematic view showing relative positional relationships of the components of the light emitting device according to a variant embodiment of the disclosure.

FIG. 5 shows the selection rule of optical transition in GaAs based semiconductor quantum wells or quantum dots.

FIG. 6 illustrates the magnetization switching in the injector Hall-bar structure by spin Hall effect (SHE).

FIG. 7 is a cross-sectional view of the light emitting device according to an embodiment of the disclosure.

FIGS. 8A and 8B are exemplified top views of the light emitting device according to an embodiment of the disclosure.

FIG. 9 is a cross-sectional view of the light emitting device according to a variant embodiment of the disclosure.

FIG. 10 is a cross-sectional view of the light emitting device according to another variant embodiment of the disclosure.

FIG. 11 is a cross-sectional view of the spin-based photodiode according to an embodiment of the disclosure.

FIG. 12 is a top view of the spin-based photodiode according to an embodiment of the disclosure.

FIG. 13 is a cross-sectional view of the spin-based photodiode according to another embodiment of the disclosure.

FIG. 14 is a cross-sectional view of the spin-based photodiode according to a variant embodiment of the disclosure.

FIGS. 15A and 15B are exemplified top views of the spin-based photodiode according to another embodiment of the disclosure.

FIG. 16 is a timing chart of an exemplified operation flow of the optical detection apparatus according to an embodiment of the disclosure.

FIG. 17 is a timing chart of an exemplified operation flow of the optical detection apparatus according to another embodiment of the disclosure.

### DETAILED DESCRIPTION

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

In the disclosure, a highly compact polarization source for biological application is provided by combining the circularly polarized emitter (spin-LED (spin Light Emitting Diode) or spin-VCSEL (spin Vertical-Cavity Surface-Emitting Laser)) and the polarization sensitive detector (spin-PD (spin-based photodiode)) without using any optical component such as ¼ waveplate or polarizer.

Spin-based light emitting device such as spin-light emitting diodes (spin-LEDs) and spin-vertical cavity surface emitting lasers (spin-VCSELs) can convert the carrier spin information to circularly polarized light to act as a compact source of circularly polarized light. The same structure of spin LED can be also inversely functionalized as a spin-based photodiode (spin-PDs) to detect the polarization of light through spin polarized current.

By illuminating an object to be detected by the light emitted by the spin-based light emitting device and detecting the light scattered back from the object by the spin-based photodiode, and analyzing the difference between the circular polarization state of the light detected by the spin-based photodiode and that of the light emitted by the spin-based light emitting device, structural information of the object can be obtained accordingly.

The spin-based light emitting device and the spin-based photodiode can be integrated at the tip of a biopsy probe apparatus such as an endoscope, enable in vivo noninvasive cancer detection while avoiding the unexpected risks associated with administering a fluorescent agent.

Embodiments of the disclosure using a combination of spin light emitting device and spin photodiode can provide the following advantages: (1) compact resource without optical components; (2) fast electrical modulation of circular polarizations compared to traditional photoelastic modulator; (3) high density of arrays of spin-LED or spin-VCSEL and spin-PD capable of being fabricated on one single wafer; and (4) low consumption of energy owing to the high quantum efficiency of LED.

### 1. Optical Detection Apparatus

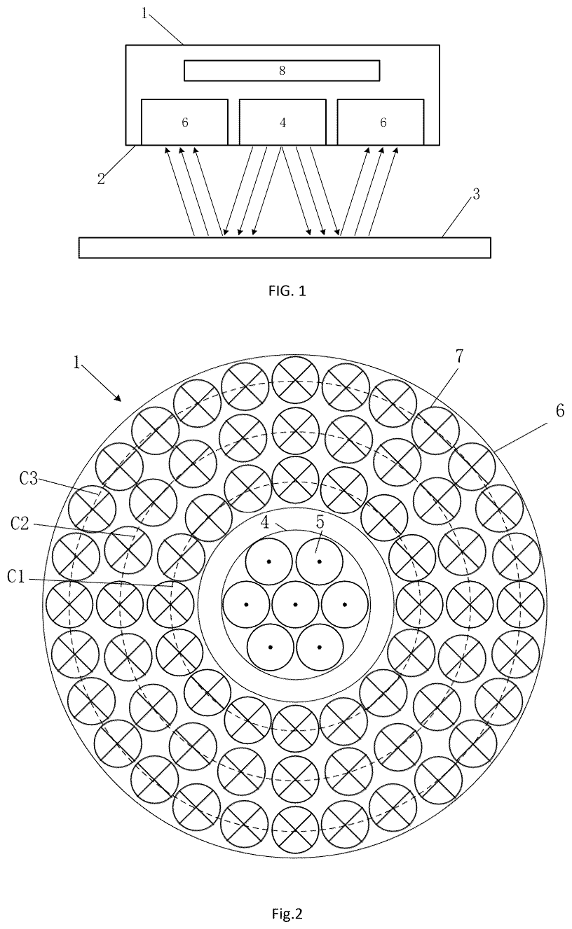

FIG. 1 is a cross-sectional view of the optical detection apparatus according to an embodiment of the disclosure. FIG. 2 is a schematic view of the arrangement of spin light emitting devices and spin photodiodes in the detection side of the optical detection apparatus.

As shown in FIGS. 1 and 2, the optical detection apparatus 1 includes a light emitting portion 4 and a light detection portion 6.

The optical detection apparatus 1 has a detection side 2, as shown in FIG. 1. From the detection side 2, the light emitting portion 4 emits light, and the light detection portion 6 receives light back from an object 3 (to be detected) reflecting the light emitted by the light emitting portion 4.

View from the detection side 2, as shown in FIG. 2, the light emitting portion 4 is arranged in the central portion, and the light detection portion 6 is arranged surrounding the light emitting portion 4.

As shown in FIG. 2, at least one light emitting device 5 (each shown as a circle with a dot) is arranged in the light emitting portion 4. In the embodiment shown in FIG. 2, 7 light emitting devices are arranged in the light emitting portion 4. The number and arrangement of the light emitting devices 5 in the light emitting portion 4 may be varied and are not limited to the embodiment shown in FIG. 2.

A plurality of photodiodes 7 (each shown as a circle with a cross) are arranged in the light detection portion 6. The number and arrangement of the photo diodes 7 in the light detection portion 6 may be varied and are not limited to the embodiment shown in FIG. 2.

In the embodiment shown in FIG. 2, the plurality of photodiodes 7 can be grouped into three sets, each arranged along an imaginary circle: a first set along a first imaginary circle C1, a second set along a second imaginary circle C2, and a third set along a third imaginary circle C3.

The imaginary circles share a common center point, which is approximately the center of the light emitting portion 4.

Different sets of photodiodes 7 are then respectively arranged to detect light reflected back from the object 3 at varying reflective angles. An inner set of photodiodes 7 arranged along a smaller imaginary circle (e.g. C1) detect light at a smaller reflective angle, and an outer set of photodiodes 7 arranged along a larger imaginary circle (e.g. C3) detect light at a larger reflective angle.

The light emitting device 5 is a spin-based light emitting device configured to emit a circularly polarized light towards the object 3 to be detected. The spin-based light emitting device 5 is a surface-emitting device having a first surface for emitting light. As shown in FIGS. 1 and 2, the first surface is arranged on the detection side 2 of the optical detection apparatus.

The spin-based light emitting device 5 can be referred to as a “vertical-type spin-based light emitting device with surface emitting geometry”. The configuration of such a spin-based light emitting device will be described in detail later in this disclosure.

The photodiodes 7 are spin-based photodiodes configured to detect the polarization state of light scattered back from the object 3. the spin-based photodiodes 7 are surface-illuminated photodiodes, each having a second surface for receiving the light scattered back from the object. As shown in FIGS. 1 and 2, the second surface is arranged on the detection side 2 of the optical detection apparatus.

The spin-based photodiode 7 can be referred to as a “vertical-type spin-based photodiode with surface illuminated geometry”. The configuration of such a spin-based photodiode will be described in detail later in this disclosure.

As shown in FIG. 1, the optical detection apparatus 1 may further include a processor 8. The processor 8 is configured to determine the circular polarization rate of the light beams received by the plurality of spin-based photodiodes 7.

In some embodiments, the processor 8 can be used to analyze the difference between the circular polarization state of the light detected by the spin-based photodiode 7 and that of the light emitted by the spin-based light emitting device 5, so as to derive the structural information of the object accordingly.

In some embodiments, the processor 8 can be used to readjust the emission intensity of the spin light emitting device, spin-orbit torque (SOT) injector modulation function, etc. Furthermore, the processor 8 can also be used to realize the function of communication with external instruments.

The light emitting portion 4 emits circularly polarized light towards an object to be detected. The circularly polarized light scattered back from the object will have changed circular polarization state. By determining the circular polarization rate of the light beams received by the plurality of spin-based photodiodes 7, and further analyzing the difference of the circular polarization between the scattered light from the object and the light emitted by the light emitting portion 4, the structural information of the object can be obtained accordingly.

In some embodiments, the relationship between the circular polarization rate of the light emitted by the spin-based light emitting device 5 and the magnetization state of the spin injector of the spin-based light emitting device 5 can be pre-calibrated.

In the embodiment shown in FIG. 1, the second surface is coplanar with the first surface.

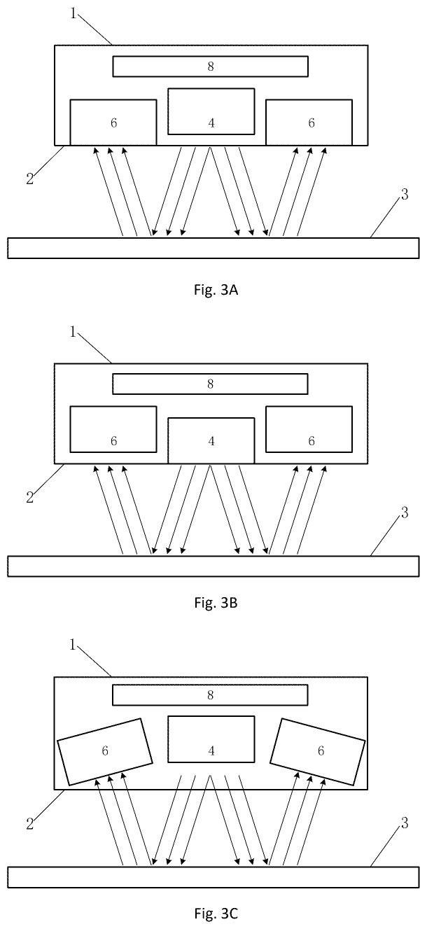

FIGS. 3A to 3C show variant arrangements of the light emitting portion and the light detection portion.

As shown in FIGS. 3A and 3B, the second surface of the photodiodes 7 in the light detection portion 6 may be arranged parallel to the first surface of the light emitting devices 5 in the light emitting portion 4. The first surface is offset backward relative to the second surface in FIG. 3A and the second surface is offset backward relative to the first surface in FIG. 3B.

As shown in FIG. 3C, the second surface of the photodiodes 7 in the light detection portion 6 may be arranged at an angle relative to the detection side 2 (or the first surface).

It shall be understood that the photodiodes 7 at different position (or different distance to the center of the light emitting portion 4) may be arranged at different angles relative to the detection side 2 (or the first surface).

In other words, a central normal line of the second surface of each photodiode 7 in the light detection portion 6 is oriented toward the region of the object 3 illuminated by the light from the light emitting devices 5 in the light-emitting portion 4.

Hereinafter, a vertical-type spin-based light emitting device with surface emitting geometry and a vertical-type spin-based photodiode with surface illuminated geometry will be described in detail.

2. Vertical-type spin-based light emitting device with surface emitting geometry

It shall be understood that the vertical-type spin-based light emitting device and the vertical-type spin-based photodiode described in this disclosure can be separately utilized for application other than optical detection apparatus 1 of the disclosure. The vertical-type spin-based light emitting device can be used to emit a circularly polarized light for applications such as telecommunication, optical computation, 3D display and so on. And, the vertical-type spin-based photodiode can be used to detect the polarization state of the received light for applications such as telecommunication, optical computation and so on.

The semiconductor spintronics technology is used to achieve the light emission with desired circular polarization. By depositing a ferromagnetic layer as a spin injection layer on the top of the quantum wells or quantum dots based LED structure, spin-polarized electrons can be injected into the semiconductor quantum wells or semiconductor quantum dots. The spin-polarized electrons will undergo quantum transition to recombine with holes according to the law of conservation of angular momentum, and thus circularly polarized photons will be emitted. Each of the semiconductor quantum wells or semiconductor quantum dots is capable of emitting photon with circular polarization direction determined by the spin direction of the injected spin-polarized carrier.

The spin-based light emitting device according to an embodiment of the disclosure includes a first multi-layer semiconductor structure and a spin injector.

The spin injector is configured to inject spin-polarized carriers into the first multi-layer semiconductor structure.

Here, the carriers can be either electrons or holes. Generally, the electrons are used as the carriers because the spin lifetime of electrons is much longer than that of holes.

In some embodiments, the first multi-layer semiconductor structure may be sandwiched between a substrate and the spin injector. When a bias voltage is applied between the spin injector and the substrate, spin-polarized carriers will be injected from the spin injector to the first multi-layer semiconductor structure.

The first multi-layer semiconductor structure includes gain medium of quantum dots (QD) or quantum wells (QW). The gain medium of quantum dots or quantum wells are capable of emitting light with circular polarization state determined by the spin direction of the injected spin-polarized carriers, in response to the spin-polarized carriers injected into the first multi-layer semiconductor structure and absorbed by the gain medium of quantum dots or quantum wells.

The spin-based light emitting device may further include a magnetic moment controller configured to control the magnetization direction of the spin injector.

It shall be understood that, the spin direction of the spin-polarized carriers injected from the spin injector into the first multi-layer semiconductor structure is determined by the magnetization direction of the spin injector.

In some embodiments, the spin injector is in a form of a bar-shaped channel.

Accordingly, the magnetic moment controller may include two electrodes respectively connected to two opposite ends of the bar-shaped channel to apply a pulsed current into the bar-shaped channel, so as to switch the magnetization direction of the spin injector. With the two electrodes, alternating reverse pulsed current may be applied into the bar-shaped channel to alternatively reverse the magnetization direction of the spin injector.

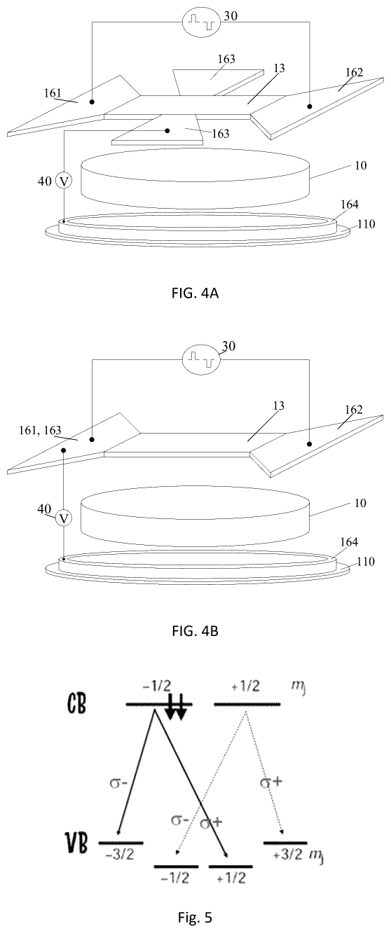

FIG. 4A is a schematic view showing relative positional relationships of the components of the light emitting device according to an embodiment of the disclosure.

As shown in FIG. 4A, a first multi-layer semiconductor structure 10 is formed above a first substrate 110, and a spin injector 13 is formed above the first multi-layer semiconductor structure 10. In other words, the first multi-layer semiconductor structure is sandwiched between the first substrate and the spin injector.

In some embodiments, the first substrate 110 is a semiconductor substrate.

In some embodiments, the first multi-layer semiconductor structure 10 is a III-V semiconductor-based structure.

In some embodiments, there might be some other layers sandwiched between the first substrate 110 and the first multi-layer semiconductor structure 10. Or, in other embodiments, the first multi-layer semiconductor structure 10 might be formed directly on the first substrate 110.

In some embodiments, there might be some other layers sandwiched between the first multi-layer semiconductor structure 10 and the spin injector 13. Or, in other embodiments, the spin injector 13 might be formed directly on the first multi-layer semiconductor structure 10.

In some embodiments, the spin injector 13 is a metallic spin injector, and may have a Hall-bar structure. the magnetization state of the spin injector can be switched by spin Hall effect (SHE).

In the example of FIG. 4A, the spin injector 13 is depicted as in a form of a bar-shaped channel.

A first electrode 161 and a second electrode 162 may be formed above the first multi-layer semiconductor structure 10 and are respectively connected to two opposite ends of the bar-shaped channel (spin injector 13). Further, the first electrode 161 and the second electrode 162 are respectively connected to two output terminals of a current pulse supplier to receive current pulses to apply current pulse into the bar-shaped channel (spin injector 13) to electrically control the magnetization direction of the spin injector 13.

As mentioned above, the spin direction of the spin-polarized carriers injected from the spin injector 13 into the first multi-layer semiconductor structure 10 is be determined by the magnetization state of the spin injector 13.

To sum up, the magnetization state of the spin injector 13 can be electrically controlled by applying current pulse into the spin injector 13 via the first electrode 161 and the second electrode 162, the spin direction of the spin-polarized carriers injected into the first multi-layer semiconductor structure 10 is thus determined, and accordingly, the circular polarization direction of the light emitted by the light emitting device 5 is determined. In other words, the circular polarization direction of the light emitted by the light emitting device 5 can be alternated by changing the direction of the current pulse applied between the first electrode 161 and the second electrode 162 and flowing through the spin injector 13.

In this example, as shown in FIG. 4A, the first electrode 161 and the second electrode 162 are respectively connected to the two opposite ends in the lengthwise direction of the bar-shaped channel (spin injector 13), so as to introduce the current pulse into the bar-shaped channel to flow through the lengthwise direction.

Further, a third electrode 163 and a fourth electrode 164 are respectively connected to two output terminals of a voltage source supplier 40 to receive voltage signals.

The third electrode 163 may be formed above the first multi-layer semiconductor structure 10, and is connected to the bar-shaped channel (spin injector 13), and the fourth electrode 164 may be formed on the first substrate 110. The third electrode 163 and the fourth electrode 164 are thus configured to apply a first voltage (bias voltage) between the spin injector 13 and the first substrate 110 to inject carriers from the spin injector 13 into the first multi-layer semiconductor structure 10.

In the example of FIG. 4A, two of the third electrodes 163 are shown on two opposite sides in widthwise direction of the bar-shaped channel (spin injector 13). It shall be understood that the third electrode 163 can be formed in many other forms, as long as it is electrically connected to the spin injector 13.

In some embodiments, one or both of the first electrode 161 and the second electrode 162 can serve as the third electrode 163 to apply voltage signal to the spin injector 13. In other words, the first electrode 161 and/or the second electrode 162 can be further connected to one output terminal of the voltage source supplier 40 to receive the voltage signals, in addition to receiving the current pulses.

FIG. 4B is a schematic view showing relative positional relationships of the components of the light emitting device according to a variant embodiment of the disclosure. As shown in FIG. 4B, the third electrode 163 and the first electrode 161 share the same electrode. The first electrode 161 further acts as the third electrode 163, connects to the voltage source supplier 40 to apply voltage signal to the spin injector 13.

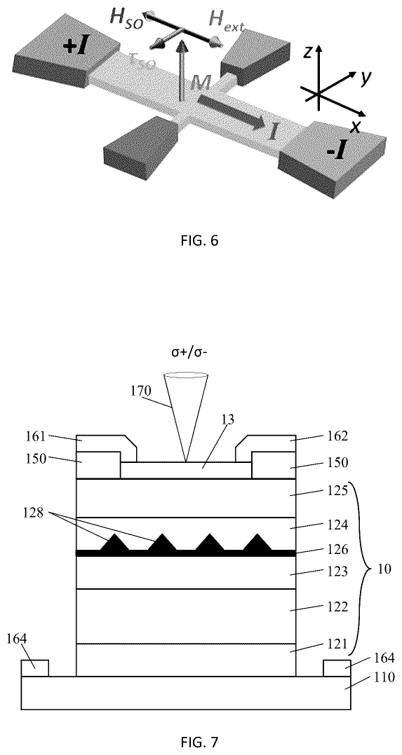

Hereinafter, the selection rule of optical transition and the magnetization switching in the spin injector will be described with reference to FIGS. 5 and 6.

FIG. 5 shows the selection rule of optical transition in GaAs semiconductor quantum wells or quantum dots.

As shown in FIG. 5, when an electron with spin of −½ is injected into the conduction band of the semiconductor quantum wells or quantum dots through a ferromagnetic spin injection layer or spin injector (such as CoFeB/MgO layer), according to the conservation law of angular momentum quantum number mⱼ (the change of angular momentum quantum number before and after the transition Δmⱼ=+1), the electron is allowed to transition to the valence band only in two ways.

One way is to transition with a heavy hole valence band (mⱼ=−3/2), that is, to transition from mⱼ=−½ to mⱼ=−3/2 (Δmⱼ=−1), emitting a left circularly polarized photon, which can be referred to as “σ−”.

The other way is to transition with a light hole valence band (mⱼ=+½), that is, to transition from mⱼ=−½ to mⱼ=+½ (Δmⱼ=+1), emitting a right circularly polarized photon, which can be referred to as “σ+”.

However, in the quantum well structure or the quantum dot structure, the light and heavy hole valence bands are non-degenerated, and the heavy hole transition matrix element (transition probability) is much higher than the light hole transition matrix element (transition probability). Therefore, while an electron with spin of −½ is injected, a left circularly polarized photon (σ−) will be obtained with almost 100% probability.

Conversely, while an electron with spin of +½ is injected, a right circularly polarized photon (σ+) will be obtained with almost 100% probability.

Therefore, the circular polarization direction of the photon emitted from the quantum wells or quantum dots completely depends on the spin direction of the injected electron. And thus, the circular polarization direction of the light beam emitted from the first multi-layer semiconductor structure 10 corresponds to the spin polarization direction of the carriers injected into the first multi-layer semiconductor structure 10.

It should be emphasized here that the optical selection rule requires the spin direction to be parallel to the photon emission direction. To obtain a circularly polarized photon without magnetic field, the magnetization direction of the ferromagnetic injection layer (spin injector 13) shall be perpendicular to the sample surface for surface emission geometry.

Based on the above principle, the first multi-layer semiconductor structure 10 is capable of emitting light beam with controllable circular polarization direction by injecting carriers with controlled spin polarization direction into the first multi-layer semiconductor structure 10.

FIG. 6 illustrates the magnetization switching in the spin injector with a Hall-bar structure by SHE.

As shown in FIG. 6, a current/is injected in the designed ferromagnet (FM)/heavy metal (HM) spin-injector channel to generate current-induced spin-orbit torque (SOT) τ_{SO} and associated spin-orbit field H_{SO}. With a small in-plane external constant magnetic field Hₑₓₜ, the perpendicular magnetization of injector can be deterministically switched when injecting alternative direction of current in the channel. The magnetization switch can be realized with very short pulse current (6ps), which allows for high-speed operation. The latest developments in the field of spintronics show the possibility to avoid the application of Hₑₓₜ by using different strategies, such as using spin textured ferromagnetic layer, an in-plane exchange bias or growth on substrates with specific crystalline orientation.

To electrically control the circular polarization direction of the emitted light beam, a current pulse will be applied to the spin injector 13 (through the first electrode 161 and the second electrode 162) to switch the magnetization state of the spin injector 13. Then, the spin injector 13 is negatively biased (through the third electrode 163 and the fourth electrode 164) to enable a light emission.

As the magnetization state changes in response to the application of the pulsed current, the spin polarization direction of the carriers injected from the spin injector 13 will change accordingly.

According to the optical selection rule, the circular polarization direction (right circular polarization σ+ or left circular polarization σ−) of the light emitted from the first multi-layer semiconductor structure 10 will be determined by the spin polarization direction of carriers injected from the spin injector 13. Therefore, by switching the magnetization state of the spin injector 13, the spin polarization direction of the injected carriers can be changed, and the circular polarization direction of the emitted light beam will be controlled accordingly.

In embodiments, the magnetization state of the spin injector 13 refers to the magnetization direction of the spin injector 13.

Magnetization direction of the spin injector 13 are flipped by applying the current pulse into the bar-shaped channel of the spin injector 13.

The magnetization state of the spin injector 13 are non-volatile and are capable of being retained after the current pulse are applied.

The parameters of the current pulse, such as amplitude, duration, numbers of sub-pulse and so on, can be configured to make sure that the magnetization direction of the whole spin injector 13 is flipped into one magnetization direction, for example, up-direction (⬆).

While a bias voltage (first voltage) is applied between the third electrode 163 and the fourth electrode 164, spin-polarized carriers are injected from the spin injector 13 to the semiconductor quantum wells or semiconductor quantum dots 128 in the first multi-layer semiconductor structure 10.

When the spin injector 13 has an up-direction (⬆) magnetization, the majority of carriers injected from the spin injector 13 into the first multi-layer semiconductor structure 10 are polarized to spin of +½. Accordingly, the light emitted from the first multi-layer semiconductor structure 10 has a positive circular polarization, in which the proportion of the right circularly polarized light (σ+) component is greater than that of the left circularly polarized light component (σ−).

And, when the spin injector 13 has a down-direction (⬇) magnetization, the majority of carriers injected from the spin injector 13 into the first multi-layer semiconductor structure 10 are polarized to spin of −½. Accordingly, the light emitted from the first multi-layer semiconductor structure 10 has a negative circular polarization, in which the proportion of the right circularly polarized light (+) component is smaller than that of the left circularly polarized light component (σ−).

In addition, after a light beam is emitted from the first multi-layer semiconductor structure 10 with the spin injector 13 in one magnetization direction, the magnetization direction of the spin injector 13 can be flipped to the opposite direction, for example, from up-direction (⬆) magnetization to down-direction (⬇) magnetization, by applying a further current pulse with a direction opposite to the previous one.

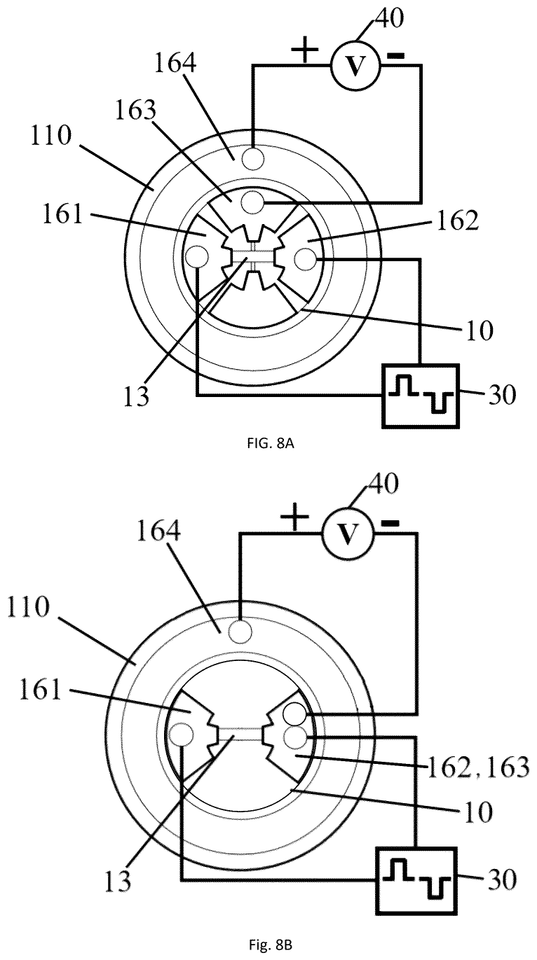

A more detailed structure of the light emitting device 5 will be described below with reference to FIG. 7, FIG. 8A and FIG. 8B.

FIG. 7 is a cross-sectional view of the light emitting device according to an embodiment of the disclosure. FIGS. 8A and 8B are exemplified top views of the light emitting device according to the embodiment of the disclosure.

As shown in FIG. 7, the light emitting device 5 includes a first multi-layer semiconductor structure 10 and a spin injector 13.

In some embodiments, the first multi-layer semiconductor structure 10 is a GaAs based structure. The first multi-layer semiconductor structure 10 can be a light emitting diode structure with a gain medium layer 128 formed above a wetting layer 126. Quantum wells or quantum dots are formed in the gain medium layer 128. P-doped layers (121, 122) are formed below the gain medium layer 128. And n-doped layer (125) is formed above the gain medium layer 128.

As shown in FIG. 7, a first multi-layer semiconductor structure 10 is formed on a first substrate 110. The first substrate 110 might be a p-doped GaAs substrate with a (001) crystal plane, i.e., a p-GaAs (001) substrate.

The first multi-layer semiconductor structure 10 includes, from bottom to top, a p-doped GaAs (p-GaAs) layer 121 (for example, 300 nm), a p-doped Al_{0.3}Ga_{0.7}As layer 122 (for example, 400 nm), a Be δ-doping layer 123 (for example, 30 nm), a wetting layer 126 of InGaAs, a gain medium layer 128 with quantum wells or quantum dots formed therein, a GaAs layer 124 (for example, 50 nm) and a n-doped GaAs layer 125 (for example, 50 nm).

In some embodiments, the first multi-layer semiconductor structure 10 is cylindrical, and when viewed from top down, it might be disc-shaped.

A spin injector 13 is formed on the top layer of the first multi-layer semiconductor structure 10, i.e., the n-doped GaAs layer 125.

As described above, the spin injector 13 might be in form of a bar. The sizes (length and width) of the upper surface of the bar-shaped spin injector 13 are smaller than the radius of upper surface of the cylindrical first multi-layer semiconductor structure 10.

As shown in FIG. 8A, a first electrode 161 and a second electrode 162, as well as a third electrode 163 are formed surrounding the spin injector 13, and connected with the spin injector 13.

The fourth electrode 164 is formed on the first substrate 110. The fourth electrode 164 might be a ring shape surrounding the cylindrical first multi-layer semiconductor structure 10 with an interval.

As described above, the first electrode 161 and the second electrode 162 are respectively connected to two opposite ends of the bar-shaped channel of the spin injector 13 to apply current pulses into bar-shaped channel of the spin injector 13.

The first electrode 161 and the second electrode 162 are further connected to a current pulse generator (or a current pulse source) 30. The current pulse generator 30 provides the current pulses with a direction corresponding to the circular polarization direction of the light beam desired to be emitted. And the current pulse generator 30 is capable of alternatively reversing the direction of the current pulses to change the circular polarization direction of the emitted light beam.

The third electrode 163 and the fourth electrode 164 are configured to apply a bias voltage (first voltage) between the spin injector 13 and the first substrate 110. A first voltage source supplier 40 may be configured to provide the bias voltage (V).

In some embodiments, the first electrode 161 and/or the second electrode 162 may also serve as the third electrode 163 to receive the bias voltage with respect to the fourth electrode 164. In other words, the first electrode 161 and/or the second electrode 162 can be further connected to one output terminal of the voltage source supplier 40 to receive the voltage signals, in addition to receiving the current pulses.

As shown in FIG. 8B, the third electrode 163 and the second electrode 162 share the same electrode. The second electrode 162 further acts as the third electrode 163, connects to the voltage source supplier 40 to apply voltage signal to the spin injector 13.

An insulating material layer 150 is formed between the electrodes 161, 162, 163 and the top layer of the first multi-layer semiconductor structure 10 and surrounding the spin injector 13, insulating the electrodes 161, 162 and 163 from the top layer of the first multi-layer semiconductor structure 10. The insulating material layer 150 might be formed by SiO₂.

In some embodiments, the first electrode 161, the second electrode 162, the third electrode 163 and the fourth electrode 164 are formed from Ti, or Au or combination of Ti and Au such as double-layer film (Ti/Au) and Ti—Au alloy.

By applying the current pulse into the spin injector 13 via the first electrode 161 and the second electrode 162, the magnetization state (magnetization direction) of the spin injector 13 will change accordingly.

And then, by applying a bias voltage (first voltage) between the spin injector 13 and the first substrate 110 via the third electrode 163 and the fourth electrode 164, spin-polarized carriers will be injected from the spin injector 13 into the first multi-layer semiconductor structure 10, especially into the quantum wells or quantum dots (gain medium layer 128).

In response to the spin-polarized carriers, the quantum wells or quantum dots 128 emit light with circular polarization (σ+ or σ−) corresponding to the spin polarization of the carriers.

And thus, the first multi-layer semiconductor structure 10 will emit a light beam 170 with right circularly polarized portions (σ+) or left circularly polarized portions (σ−).

As mentioned above, the light emitting device as shown in FIG. 7 may be referred to “spin-LED”.

The first multi-layer semiconductor structure 10 may be in a form of multi-layer mesa. FIGS. 7, 8A and 8B show only one light emitting device 5 formed on one multi-layer mesa with one spin injector 13.

In some embodiments, a plurality of light emitting devices 5 can be formed on one multi-layer mesa. The plurality of light emitting devices 5 share the multi-layer semiconductor mesa as the first multi-layer semiconductor structure 10.

In a variant embodiment, a bottom distributed Bragg reflector (DBR) may be formed under the first multi-layer semiconductor structure 10.

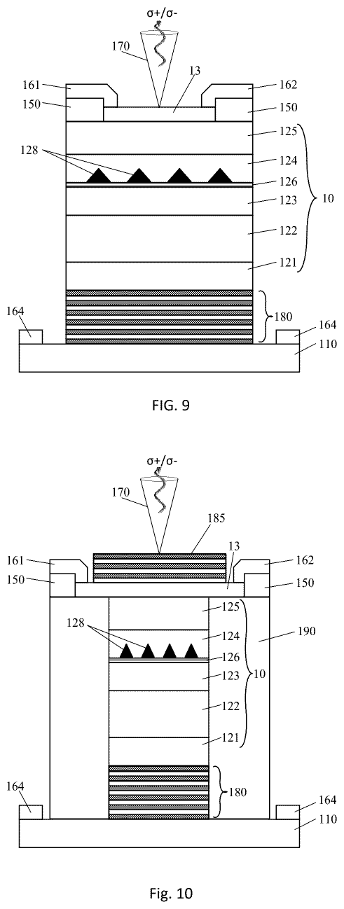

FIG. 9 is a cross-sectional view of the light emitting device according to a variant embodiment of the disclosure. The light emitting device shown in FIG. 9 is a spin-LED with bottom DBR to enhance the light intensity.

As compared with the embodiment as shown in FIG. 7, in the variant embodiment shown in FIG. 9, a bottom distributed Bragg reflector (DBR) 180 is arranged below the first multi-layer semiconductor structure 10. In other words, the first multi-layer semiconductor structure 10 is sandwiched between the spin injector 13 and the bottom distributed Bragg reflector (DBR) 180.

By arranging the bottom distributed Bragg reflector (DBR) 180 below the first multi-layer semiconductor structure 10, the light output efficiency in the upward direction can be enhanced.

The Distributed Bragg reflector (DBR) is a periodic structure composed of two materials with different refractive index arranged alternately in the stack form of ABAB. The product of reflection index and the thickness of each layer of material is equal to ¼ of the central reflection wavelength.

The bottom distributed Bragg reflector (DBR) 180 may be formed as a dielectric Bragg reflector structure by alternative deposition of different materials such as AlGaAs/AlAs.

The light emitting device as shown in FIG. 9 can be referred to as “spin ½-VCSEL”. In another variant embodiment, a spin-VCSEL can be formed.

FIG. 10 is a cross-sectional view of the light emitting device according to another variant embodiment of the disclosure. The light emitting device as shown in FIG. 10 can be referred to as “spin-VCSEL”, an optical cavity is formed between a bottom DBR and a top DBR.

The optical cavity provides several unique advantages: (1) lasing threshold reduction, i.e., the lasing threshold is reduced compared to conventional lasers; and (2) spin amplification, i.e., the circular polarization (Pc) of the light emitted from the light emitting device 5 is greater than the spin polarization (Pn) of the carriers injected from the spin injector 13. Remarkably, for injection between majority and minority spin thresholds, J_{T1}<J<J_{T2}, even a small spin polarization (Pₙ) will lead to circular polarization rate (Pc) of approximately 100%. Indeed, experiments yield circular polarization rate (Pc) of about 96% for spin polarization (Pn) of about 4%.

As compared with the embodiment as shown in FIG. 9, in the variant embodiment as shown in FIG. 10, a top distributed Bragg reflector (DBR) 185 is arranged above the spin injector 13. In other words, the spin injector 13 is sandwiched between the first multi-layer semiconductor structure 10 and the top distributed Bragg reflector (DBR) 185.

By further arranging a top distributed Bragg reflector (DBR) 185 above the spin injector 13, an intracavity resonant surface emitting laser structure is formed between the top distributed Bragg reflector (DBR) 185 and the bottom distributed Bragg reflector (DBR) 180. And, a Vertical-Cavity Surface Emitting Laser (VCSEL) is thus formed. Such a micro-scale resonant cavity can greatly improve the light emission efficiency of the quantum wells or the quantum dots.

The top distributed Bragg reflector (DBR) 185 may be formed as a dielectric Bragg reflector structure by alternative deposition of different materials such as TiO₂/Al₂O₃, CaF₂/ZnS, MgF₂/ZnS.

In order to form a spin-VCSEL structure (FIG. 10), the semiconductor part, which includes the bottom DBR 180 and the first multi-layer semiconductor structure 10 with an embedded gain medium (quantum wells or quantum dots), is firstly formed on the first substrate 110. And then, the semiconductor part is surrounded by photoresist 190 (for example, benzocyclobutene (BCB)) with a flat surface.

The spin injector 13 will then be deposited on the top surface of the semiconductor part and the photoresist 190 and followed by lithography process to form the Hall-bar structure. The surface area of the spin injector 13 is large enough to cover all the first multi-layer semiconductor structure 10 to ensure a homogenous current injection into the gain medium region for laser emission.

To minimize the optical absorption risk due to the inserted metallic spin injector 13 in the cavity, the distance between the spin injector 13 and the active region (i.e. the gain medium of quantum dots or quantum wells) is configured so that the spin injector 13 is placed in one node of the stationary electromagnetic field formed by the light reflected from the top and bottom distributed Bragg reflectors.

The top DBR 185 is then deposited on the spin injector 13 to complete the optical cavity.

Pulsed current will be sent into the spin injector via the first electrode 161 and the second electrode 162 to switch the magnetization state of spin injector 13. The spin injector 13 will be negatively biased by the third electrode 163 and the fourth electrode 164 for a continuous emission. σ+ and σ-will be modulated according to the pulsed-current direction in the channel of spin injector 13.

3. Vertical-Type Spin-Based Photodiode with Surface Illuminated Geometry

The spin-based photodiode may have similar structure as the spin-LED shown in FIG. 7 and FIG. 8A/8B, while careful design of active region should be taken account in order to enhance the helicity detection efficacy. The bandgap of the spin-based photodiode should be slight smaller than the bandgap of active region material in the spin-LED or spin-VCSEL. The spin detector in the spin-based photodiode may have the same FM/HM structure as spin injector in the spin-LED or spin-VCSEL. However, in order to enhance the tunneling transmission, the tunneling barrier thickness in the spin detector might be configured as thin as possible (still should be thick enough to support perpendicular magnetic anisotropy of FM).

In some embodiments, the spin detector of the spin-based photodiode might have the same circle size as the mesa (or, the multi-layer semiconductor structure with the gain medium of quantum wells or quantum dots formed therein). In some other embodiments, the spin detector of the spin-based photodiode might have a bar-shaped structure similar to the spin injector 13 as shown in FIGS. 8A and 8B.

The spin-based photodiode may have a second multi-layer semiconductor structure with the same structure as that of the first multi-layer semiconductor structure in the spin-based light emitting device (spin-LED or spin-VCSEL). A gain medium of quantum wells or quantum dots is also formed in the second multi-layer semiconductor structure.

With the illumination by the circularly polarized light, the light will create spin polarized carriers in the multi-layer semiconductor structure with the gain medium of quantum wells or quantum dots formed therein.

With the drift field by a bias voltage, the spin-polarized carriers will move to the interface between the spin detector and the semiconductor (SC) structure.

The measured photo-generated current in the spin-PD will depend on the relative direction between the magnetization direction of spin detector and the spin polarized photo-generated carrier. If they are parallel, a larger photocurrent will be detected. If they are antiparallel, a smaller photocurrent will be detected. Therefore, depending on the magnetization direction of spin detector, the amplitude of the measured spin photocurrent allows to extract the circular polarization rate of the incident light.

In some embodiments, the correlative relationship between the circular polarization rate of the light detected by the spin-based photodiode 7 and the amplitude of the measured spin photocurrent can be pre-calibrated.

In some embodiments, by using spin-orbit torque (SOT) detector, the magnetization of spin detector can be switched. Instead of measuring an absolute value of spin photocurrent, a difference between the spin photocurrents respectively derived under two opposite detector magnetization directions can be measured. This allows an increase in the accuracy of circular polarization rate (Pc) measurement.

When the polarized light is reflected or scattered from the biological tissue surface, the polarization state will be modified depending the status of tissue (normal or cancerized). Therefore, we are able to detect the healthy information by using the combination of the spin-LED (spin-VCSEL) and the spin-based photodiode.

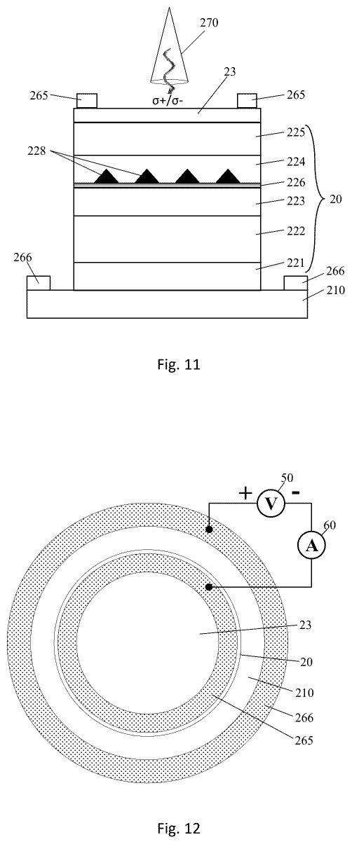

FIG. 11 is a cross-sectional view of the spin-based photodiode according to an embodiment of the disclosure. FIG. 12 is a top view of the spin-based photodiode according to an embodiment of the disclosure.

As shown in FIGS. 11 and 12, the spin-based photodiode 7 includes a second substrate 210, a second multi-layer semiconductor structure 20 and a spin detector 23.

The second multi-layer semiconductor structure 20 is formed above the second substrate 210. The second multi-layer semiconductor structure 20 is capable of creating spin-polarized carriers with the illumination of circularly polarized light.

In the embodiment shown in FIG. 11, the second multi-layer semiconductor structure 20 includes gain medium of quantum dots or quantum wells. The gain medium of quantum dots or quantum wells are capable of creating spin-polarized carriers with the illumination of circularly polarized light. As will described below with reference to FIG. 14, the second multi-layer semiconductor structure 20 may include a PN junction structure capable of creating spin-polarized carriers with the illumination of circularly polarized light.

The spin detector 23 is formed above the second multi-layer semiconductor structure 20. And, the spin detector 23 is capable of detecting a helicity dependent spin photocurrent flowing through the spin detector. The spin detector 23 may be formed from the same material as the spin injector 13, i.e., the spin detector 23 may be a ferromagnetic layer such as CoFeB/MgO layer. And, as described above, the spin detector 23 in the spin-based photodiode may have the same FM/HM structure as spin injector 13 in the spin-LED or spin-VCSEL.

The magnetization direction of the whole spin detector 23 is pre-magnetized to one magnetization direction, for example, up-direction (⬆).

In some embodiments, the second substrate 210 is a semiconductor substrate.

The second substrate 210 might be a p-doped GaAs substrate with a (001) crystal plane, i.e., a p-GaAs (001) substrate.

In some embodiments, the second multi-layer semiconductor structure 20 is a III-V semiconductor-based structure.

In some embodiments, the second multi-layer semiconductor structure 20 is a GaAs based structure. The second multi-layer semiconductor structure 20 can be a light emitting diode structure with a gain medium layer 228 formed above a wetting layer 226. Quantum wells or quantum dots are formed in the gain medium layer 228. P-doped layers (221, 222) are formed below the gain medium layer 228. And n-doped layer (225) is formed above the gain medium layer 228.

In some embodiments, the second multi-layer semiconductor structure 20 includes, from bottom to top, a p-doped GaAs (p-GaAs) layer 221 (for example, 300 nm), a p-doped Al_{0.3}Ga_{0.7}As layer 222 (for example, 400 nm), a Be δ-doping layer 223 (for example, 30 nm), a wetting layer 226 of InGaAs, a gain medium layer 228 with quantum wells or quantum dots formed therein, a GaAs layer 224 (for example, 50 nm) and a n-doped GaAs layer 225 (for example, 50 nm).

In some embodiments, the second multi-layer semiconductor structure 20 is cylindrical, and when viewed from top down, it might be circular disc-shaped.

A spin detector 23 is formed on the top layer of the second multi-layer semiconductor structure 20, i.e., the n-doped GaAs layer 225.

As shown in FIG. 11, the spin detector 23 may have the same surface shape as the second multi-layer semiconductor structure 20 when observed from the second surface for receiving the light. In other words, the spin detector 23 may also be circular disc-shaped.

As shown in FIGS. 11 and 12, the spin-based photodiode 7 may further includes a fifth electrode 265 connected to the spin detector 23 and a sixth electrode 266 connected to the second substrate 210.

The fifth electrode 265 and the sixth electrode 266 are configured to apply a second voltage (bias voltage (V)) between the spin detector 23 and the second substrate 210 to drive the spin-polarized carriers created from the gain medium to the spin detector 23. A second voltage source supplier 50 may be configured to provide the second voltage.

A current meter 60 is also connected to the fifth electrode 265 and the sixth electrode 266 and configured to detect the helicity dependent spin photocurrent flowing through the spin detector 23.

In some embodiments, the bandgap of the gain medium of the spin-based photodiode 7 is smaller than the bandgap of a gain medium of the spin-based light emitting device 5.

In some embodiments, the tunneling barrier in the spin detector of the spin-based photodiode 7 is thinner that the tunneling barrier in the spin injector of the spin-based light emitting device 5.

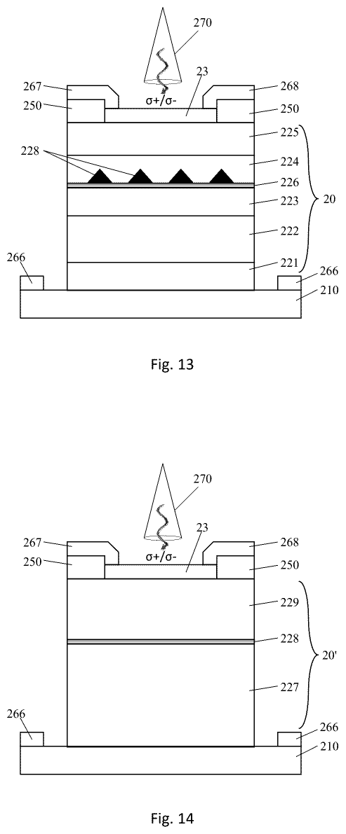

FIG. 13 is a cross-sectional view of the spin-based photodiode according to another embodiment of the disclosure.

The structure of the spin-based photodiode as shown in FIG. 13 is similar to that of the spin-based light emitting device as shown in FIG. 7.

The second substrate 210, the second multi-layer semiconductor structure 20 and the sixth electrode 266 are the same as those shown in FIG. 11.

FIG. 14 is a cross-sectional view of the spin-based photodiode according to a variant embodiment of the disclosure. As compared with FIG. 13, the second multi-layer semiconductor structure 20′ has a PN junction structure without quantum dots or quantum wells. The second multi-layer semiconductor structure 20′ includes a p-doped layer 227, a n-doped layer 229 and a PN junction region 228 formed between them. The PN junction structure is capable of creating spin-polarized carriers with the illumination of circularly polarized light.

The spin detector 23 as shown in FIGS. 13 and 14 has a different shape as compared with that of FIG. 11. The spin detector 23 of the spin-based photodiode of the embodiments shown in FIGS. 13 and 14 may be in a form of a bar-shaped channel, which is similar to the spin injector 13 as shown in FIGS. 8A and 8B, respectively. The spin-based photodiode further includes two electrodes respectively connected to two opposite ends of the bar-shaped channel of the spin detector 23 to apply a pulsed current into the bar-shaped channel of the spin detector 23, so as to switch the magnetization direction of the spin detector 23.

The principle of switching the magnetization direction of the spin detector 23 in the spin-based photodiode 7 is the same as that of the spin injector 13 in the spin-based light emitting device 5.

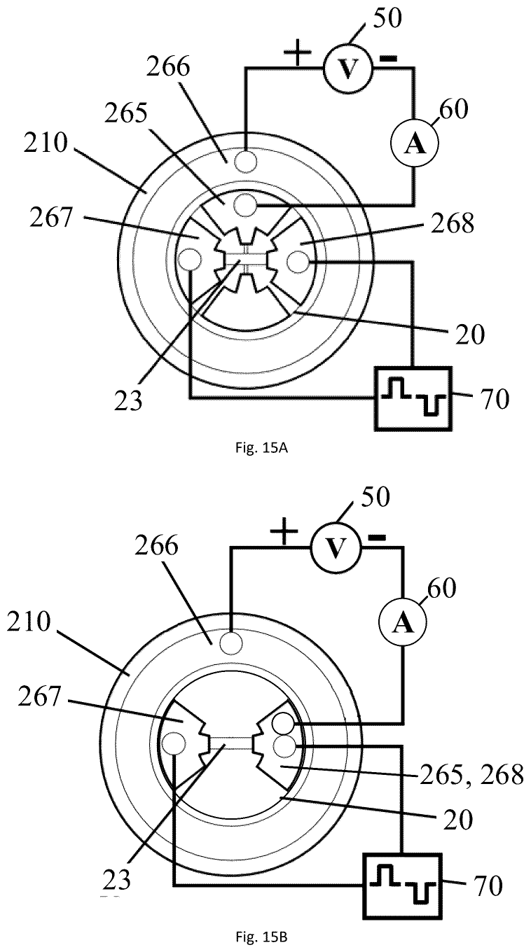

FIGS. 15A and 15B are exemplified top views of the spin-based photodiode according to the other embodiment of the disclosure.

The top views of the spin-based photodiode shown in FIGS. 15A and 15B are similar to those shown in FIGS. 8A and 8B, respectively.

As shown in FIG. 15A, the fifth electrode 265 is formed above the second multi-layer semiconductor structure 20, and connected to the spin detector 23.

The sixth electrode 266 is formed on the second substrate 210, and might be a ring shape surrounding the cylindrical second multi-layer semiconductor structure 20 (or 20′) with an interval. The connection relationships and functions of the fifth electrode 265, sixth electrode 266, the second voltage source supplier 50 and the current meter 60 are the same as those described above with reference to FIGS. 11 and 12.

A seventh electrode 267 and an eighth electrode 268 are respectively connected to two opposite ends of the bar-shaped channel of the spin detector 23 to apply a pulsed current into the bar-shaped channel of the spin detector 23, so as to switch the magnetization direction of the spin detector 23.

The seventh electrode 267 and the eighth electrode 268 are further connected to a current pulse generator (or a current pulse source) 70. The current pulse generator 70 provides the current pulses with a direction capable of switching the spin detector 23 to the desired magnetization direction. And the current pulse generator 70 is capable of alternatively reversing the direction of the current pulses to change the magnetization direction of the spin detector 23.

As shown in FIGS. 13 and 14, an insulating material layer 250 is formed between the electrodes 265, 267 and 268. The insulating material layer 250 may be the same as the insulating material layer 150 as shown in FIG. 7.

As shown in FIG. 15B, the fifth electrode 265 and the eighth electrode 268 share the same electrode. The eight electrode 268 further acts as the fifth electrode 265, connects to the voltage source supplier 50 and the current meter 60 (in serial), so as to apply voltage signal to the spin injector 13 and detect the helicity dependent spin photocurrent flowing through the spin detector 23.

Embodiments of the optical detection apparatus of the disclosure is described in detail.

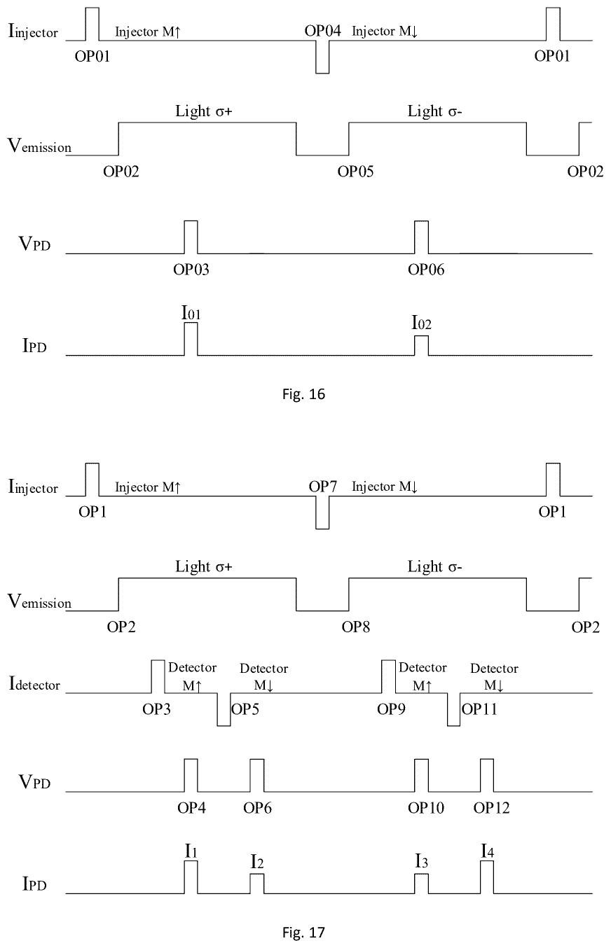

FIG. 16 is a timing chart of an exemplified operation flow of the optical detection apparatus according to an embodiment of the disclosure. Such an operation flow can be performed when the magnetization direction of the spin detectors 23 of the spin-based photodiodes 7 is pre-magnetized to a certain magnetization direction, for example, up-direction (⬆). In other words, the operation flow can be performed with spin-based photodiodes 7 of the embodiment shown in FIGS. 11 and 12. The timing flow can be controlled by the processor 8.

In FIG. 16, one round of operations (OP01 to OP06) is depicted. And the operations depicted in FIG. 16 can be performed for many rounds.

In FIG. 16, “I_{injector}” indicates the current pulse applied to the spin injector 13 via the first electrode 161 and the second electrode 162 in the spin-based light emitting device 5; “Vₑₘᵢₛₛᵢₒₙ” indicates the first voltage applied via the third electrode 163 and the fourth electrode 164 in the spin-based light emitting device 5 for light emission; “V_{PD}” indicates the second voltage applied via the fifth electrode 265 and the sixth electrode 266 in the spin-based photodiode by the voltage source supplier 50. “IPD” indicates the amplitude of the photocurrent detected by the current meter 60.

In operation OP01, a positive current pulse of I_{injector} is applied into the spin injector 13 of the spin-based light emitting device 5. The magnetization direction of the spin injector 13 is flipped into a first direction, for example, up-direction (⬆).

In operation OP02, a first voltage Vₑₘᵢₛₛᵢₒₙ is applied between the spin injector 13 and the first substrate 110 for a first time period. Light with a right circular polarization (σ+) is emitted accordingly from the spin-based light emitting device 5 during the first time period.

In operation OP03, a second voltage V_{PD} is applied between the spin detector 23 and the second substrate 210 for a short time period, a photocurrent IPD is detected and the result is I₀₁.

The result I₀₁ photocurrent I_{PD} is detected during the first time period in which light with a right circular polarization (σ+) is emitted.

After the expiration of the first time period, in operation OP04, a negative current pulse of I_{injector} is applied into the spin injector 13 of the spin-based light emitting device 5. The magnetization direction of the spin injector 13 is flipped into a second direction opposite to the first direction, for example, down-direction (⬇).

In operation OP05, a first voltage Vₑₘᵢₛₛᵢₒₙ is applied between the spin injector 13 and the first substrate 110 for a second time period. The second time period may have the same length as the first time period. Light with a left circular polarization (σ−) is emitted accordingly from the spin-based light emitting device 5 during the second time period.

The left circularly polarized light emitted in the second time period and the right circularly polarized light emitted in the first time period have the same absolute value of circular polarization rate but opposite signs. For example, if the right circularly polarized light has a circular polarization rate of +60%, the left circularly polarized light will have a circular polarization rate of −60%.

In operation OP06, a second voltage is applied between the spin detector 23 and the second substrate 210 for a short time period, a photocurrent I_{PD} is detected and the result is I₀₂.

The result I₀₂ of photocurrent I_{PD} is detected during the second time period in which light with a left circular polarization (σ−) is emitted.

With the two results obtained in one round of operations, I₀₁ and I₀₂, an asymmetry factor Asy of spin polarized photocurrent can be derived as follow:

$$Asy = 2 \times \frac{{I}_{01} - {I}_{02}}{{I}_{01} + {I}_{02}}$$

The value of circular polarization rate (Pc) is difficult to be measured directly. However, the asymmetry factor Asy is proportional to the value of circular polarization rate (Pc).

The asymmetry factor Asy can be pre-calibrated by measuring the value of asymmetry factor Asy while illuminating light with a circular polarization rate (Pc) of 100% on the light receiving surface (the second surface) of the spin-based photodiode. Then, the circular polarization rate (Pc) of the light detected by the spin-based photodiode 7 can be obtained by determining the asymmetry factor Asy.

FIG. 17 is a timing chart of an exemplified operation flow of the optical detection apparatus according to another embodiment of the disclosure. Such an operation flow can be performed when the magnetization direction of the spin detectors 23 of the spin-based photodiodes 7 are switchable. In other words, the operation flow can be performed with spin-based photodiodes 7 of the embodiments shown in FIGS. 13 to 15B. The timing flow can be controlled by the processor 8.

In FIG. 17, one round of operations (OP1 to OP12) is depicted. And the operations depicted in FIG. 17 can be performed for many rounds.

In FIG. 17, “I_{injector}” indicates the current pulse applied to the spin injector 13 via the first electrode 161 and the second electrode 162 in the spin-based light emitting device 5; “Vₑₘᵢₛₛᵢₒₙ” indicates the first voltage applied via the third electrode 163 and the fourth electrode 164 in the spin-based light emitting device 5 for light emission; “I_{detector}” indicates the current pulse applied to the spin detector 23 via the seventh electrode 267 and the eighth electrode 268 in the spin-based photodiode 7; “V_{PD}” indicates the second voltage applied via the fifth electrode 265 and the sixth electrode 266 in the spin-based photodiode by the voltage source supplier 50. “I_{PD}” indicates the amplitude of the photocurrent detected by the current meter 60.

In operation OP1, a positive current pulse of I_{injector} is applied into the spin injector 13 of the spin-based light emitting device 5. The magnetization direction of the spin injector 13 is flipped into a first direction, for example, up-direction (⬆).

In operation OP2, a first voltage Vₑₘᵢₛₛᵢₒₙ is applied between the spin injector 13 and the first substrate 110 for a first time period. Light with a right circular polarization (+) is emitted accordingly from the spin-based light emitting device 5 during the first time period.

In operation OP3, a positive current pulse of I_{detector} is applied into the spin detector 23 of the spin-based photodiode 7. The magnetization direction of the spin detector 23 is flipped into a first direction, for example, up-direction (⬆), which is parallel to that of spin injector 13.

In operation OP4, a second voltage V_{PD} is applied between the spin detector 23 and the second substrate 210 for a short time period, a photocurrent I_{PD} is detected and the result is I₁.

In operation OP5, a negative current pulse of I_{detector} is applied into the spin detector 23 of the spin-based photodiode 7. The magnetization direction of the spin detector 23 is flipped into a second direction opposite to the first direction, for example, down-direction (⬇), which is antiparallel to that of spin injector 13.

In operation OP6, a second voltage V_{PD} is applied between the spin detector 23 and the second substrate 210 for a short time period, a photocurrent I_{PD} is detected and the result is I₂.

The two results I₁ and/2 of photocurrent I_{PD} are detected during the first time period in which light with a right circular polarization (+) is emitted.

After the expiration of the first time period, in operation OP7, a negative current pulse of I_{injector} is applied into the spin injector 13 of the spin-based light emitting device 5. The magnetization direction of the spin injector 13 is flipped into a second direction opposite to the first direction, for example, down-direction (⬇).

In operation OP8, a first voltage Vₑₘᵢₛₛᵢₒₙ is applied between the spin injector 13 and the first substrate 110 for a second time period. The second time period may have the same length as the first time period. Light with a left circular polarization (σ−) is emitted accordingly from the spin-based light emitting device 5 during the second time period.

The left circularly polarized light emitted in the second time period and the right circularly polarized light emitted in the first time period have the same absolute value of circular polarization rate but opposite signs. For example, if the right circularly polarized light has a circular polarization rate of +60%, the left circularly polarized light will have a circular polarization rate of −60%.

In operation OP9, a positive current pulse of I_{detector} is applied into the spin detector 23 of the spin-based photodiode 7. The magnetization direction of the spin detector 23 is flipped into a first direction, for example, up-direction (⬆), which is now antiparallel to that of spin injector 13.

In operation OP10, a second voltage V_{PD} is applied between the spin detector 23 and the second substrate 210 for a short time period, a photocurrent I_{PD} is detected and the result is I₃.

In operation OP11, a negative current pulse of I_{detector} is applied into the spin detector 23 of the spin-based photodiode 7. The magnetization direction of the spin detector 23 is flipped into the second direction, for example, down-direction (⬇), which is now parallel to that of spin injector 13.

In operation OP12, a second voltage V_{PD} is applied between the spin detector 23 and the second substrate 210 for a short time period, a photocurrent I_{PD} is detected and the result is I₄.

The two results/3 and/4 of photocurrent I_{PD} are detected during the second time period in which light with a left circular polarization (σ−) is emitted.

With the four results obtained in one round of operations, I₁, I₂, I₃ and I₄, an asymmetry factor Asy of spin polarized photocurrent can be derived as follow:

$$Asy = 2 \times \frac{[ ( {I}_{1} - {I}_{2} ) + ( {I}_{4} - {I}_{3} ) ]}{( {I}_{1} + {I}_{2} + {I}_{3} + {I}_{4} )}$$

The value of circular polarization rate (Pc) is difficult to be measured directly. However, the asymmetry factor Asy is proportional to the value of circular polarization rate (Pc).

The asymmetry factor Asy can be pre-calibrated by measuring the value of asymmetry factor Asy while illuminating light with a circular polarization rate (Pc) of 100% on the light receiving surface (the second surface) of the spin-based photodiode. Then, the circular polarization rate (Pc) of the light detected by the spin-based photodiode 7 can be obtained by determining the asymmetry factor Asy.

It shall be understood that, by using a bar-shaped spin detector as shown in FIGS. 13 to 15B, a more precise result can be obtained than a circular-shaped spin detector as shown in FIGS. 11 and 12. However, the circular-shaped spin detector as shown in FIGS. 11 and 12 has larger area for receiving light, and will be more sensitive for weak light intensity than the bar-shaped spin detector as shown in FIGS. 13 to 15B.

The optical detection apparatus of the disclosure can be utilized in various application, especially medical applications.

The optical detection apparatus of the disclosure can be incorporated in an endoscopic tip device to perform optical detection.

Also, the optical detection apparatus of the disclosure can be incorporated in a biomedical monitoring device configured to be embedded inside human or animal body for real-time observation on a specific area.

In the above embodiments, the spin-based photodiode of the disclosure is incorporated in the optical detection apparatus. However, it shall be understood that, the vertical-type spin-based photodiode with surface illuminated geometry described in this disclosure can be used separately in various application to detect the circular polarization state of an incident light.

In this disclosure, an SOT spin-optoelectronic based highly compact polarization source is provided.

Since no optical component (such as ¼ waveplate or polarizer) is used in the optical detection apparatus, the compact apparatus can have a volume smaller than 1 mm, which is favorable to be used as endoscopy tip or other probe tip inside human or animal body for biomedical applications.

In embodiments, the spin-based light emitting device (SOT spin-LEDs and spin-VCSELs) and the spin-based photodiode have the capability to electrically modulate the magnetization direction of the spin injector and the spin detector, respectively. The modulation speed can vary from kHz to GHz range. This modulation can largely increase the polarization detection signal-to-noise ratio.

Further, since all spin-based light emitting devices and spin-based photodiodes are in surface emitting or detection geometry, a large area with high density of devices can be fabricated to construct a 2D mapping of signal.

In addition, since the spin-based light emitting devices and spin-based photodiodes share similar structure and materials, the material syntheses and device fabrication is facilitated.

Those skilled in the art may understand that appropriate modifications can be made to various above-described circuit structures of the present disclosure as needed, all of which are within the scope of protection of the present disclosure.

Various embodiments of the present disclosure have been described above, and the foregoing descriptions are exemplary, not exhaustive, and not limiting of the disclosed embodiments. Numerous modifications and variations will be apparent to those of ordinary skill in the art without departing from the scope and spirit of the described embodiments. The terminology used herein was chosen to best explain the principles of the various embodiments, the practical application or improvement over the technology in the marketplace, or to enable others of ordinary skill in the art to understand the various embodiments disclosed herein.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An optical detection apparatus comprising:
at least one spin-based light emitting device configured to emit a circularly polarized light towards an object to be detected, the spin-based light emitting device is a surface-emitting device having a first surface for emitting light; and
a plurality of spin-based photodiodes arranged around the at least one spin-based light emitting device and configured to detect the polarization state of light scattered back from the object, the spin-based photodiodes are surface-illuminated photodiodes each having a second surface for receiving the light scattered back from the object.

2. The optical detection apparatus according to claim 1 wherein
the second surface is coplanar with or parallel to the first surface; or
a central normal line of the second surface is oriented toward the region of the object illuminated by the light from at least one spin-based light-emitting device.

3. The optical detection apparatus according to claim 1, wherein the spin-based light emitting device comprises:
a first multi-layer semiconductor structure comprising gain medium of quantum dots or quantum wells, the gain medium of quantum dots or quantum wells are capable of emitting light with circular polarization state determined by the spin direction of the injected spin-polarized carriers; and
a spin injector configured to inject spin-polarized carriers into the first multi-layer semiconductor structure.

4. The optical detection apparatus according to claim 3, wherein the spin injector is in a form of a bar-shaped channel, the spin-based light emitting device further comprises:
a first electrode and a second electrode respectively connected to two opposite ends of the bar-shaped channel to apply a pulsed current into the bar-shaped channel, so as to switch the magnetization direction of the spin injector,
wherein the spin direction of the spin-polarized carriers injected from the spin injector into the first multi-layer semiconductor structure is determined by the magnetization direction of the spin injector.

5. The optical detection apparatus according to claim 4, wherein
alternating reverse pulsed current is applied into the bar-shaped channel to alternatively reverse the magnetization direction of the spin injector.

6. The optical detection apparatus according to claim 3, wherein the spin-based light emitting device further comprises:
a first substrate, wherein the first multi-layer semiconductor structure is sandwiched between the first substrate and the spin injector;
a third electrode connected to the spin injector; and
a fourth electrode connected to the first substrate,
wherein the third electrode and the fourth electrode are configured to apply a first voltage between the spin injector and the first substrate to inject carriers from the spin injector into the first multi-layer semiconductor structure.

7. The optical detection apparatus according to claim 3, wherein the spin-based light emitting device further comprises:
a bottom distributed Bragg reflector, wherein the first multi-layer semiconductor structure is sandwiched between the spin injector and the bottom distributed Bragg reflector.

8. The optical detection apparatus according to claim 7, wherein the spin-based light emitting device further comprises:
a top distributed Bragg reflector, wherein the spin injector is sandwiched between the first multi-layer semiconductor structure and the top distributed Bragg reflector, and an intracavity resonant surface emitting laser structure is formed between the top distributed Bragg reflector and the bottom distributed Bragg reflector.

9. The optical detection apparatus according to claim 8, wherein
a surface area of the spin injector is large enough to cover the first multi-layer semiconductor structure to ensure a homogenous carrier injection into the gain medium.

10. The optical detection apparatus according to claim 8, wherein:
the distance between the spin injector and the gain medium of quantum dots or quantum wells is configured to place the spin injector in one node of the stationary electromagnetic field formed by the light reflected from the top and bottom distributed Bragg reflectors.

11. The optical detection apparatus according to claim 1, wherein the spin-based photodiodes comprises:
a second substrate;
a second multi-layer semiconductor structure formed above the second substrate, the second multi-layer semiconductor structure is capable of creating spin-polarized carriers with the illumination of circularly polarized light; and
a spin detector formed above the second multi-layer semiconductor structure, and the spin detector is capable of detecting a helicity dependent spin photocurrent flowing through the spin detector.

12. The optical detection apparatus according to claim 11, wherein the spin-based photodiodes further comprises:
a fifth electrode connected to the spin detector;
a sixth electrode connected to the second substrate, wherein the fifth electrode and the sixth electrode are configured to apply a second voltage between the spin detector and the second substrate to drive the spin-polarized carriers created in the second multi-layer semiconductor structure to the spin detector; and
a current meter connected to the fifth electrode and the sixth electrode and configured to detect the helicity dependent spin photocurrent flow through the spin detector.

13. The optical detection apparatus according to claim 11, wherein
the second multi-layer semiconductor structure comprises a gain medium of quantum dots or quantum wells capable of creating spin-polarized carriers with the illumination of circularly polarized light, and a bandgap of the gain medium of the spin-based photodiode is smaller than a bandgap of a gain medium of the spin-based light emitting device; or
the second multi-layer semiconductor structure comprises a PN junction structure capable of creating spin-polarized carriers with the illumination of circularly polarized light.

14. The optical detection apparatus according to claim 11, wherein
a tunneling barrier in the spin detector of the spin-based photodiode is thinner that a tunneling barrier in the spin injector of the spin-based light emitting device.

15. The optical detection apparatus according to claim 11, wherein
the spin detector has the same surface shape as the second multi-layer semiconductor structure when observed from the second surface, or
the spin detector is in a form of a bar-shaped channel, the vertical-type spin-based photodiode further comprising a seventh electrode and an eighth electrode respectively connected to two opposite ends of the bar-shaped channel of the spin detector to apply a pulsed current into the bar-shaped channel of the spin detector, so as to switch the magnetization direction of the spin detector.

16. The optical detection apparatus according to claim 1 further comprising:
a processor configured to determine the circular polarization rate of the light beams received by the plurality of spin-based photodiodes.

17. An endoscopic tip device comprising an optical detection apparatus according to claim 1.

18. A biomedical monitoring device configured to be embedded inside human or animal body for real-time observation on a specific area, wherein the biomedical monitoring device comprises an optical detection apparatus according to claim 1.

19. A vertical-type spin-based photodiode with surface illuminated geometry comprising:
a substrate;
a multi-layer semiconductor structure formed above the substrate, the multi-layer semiconductor structure is capable of creating spin-polarized carriers with the illumination of circularly polarized light;
a spin detector formed above the multi-layer semiconductor structure, and the spin detector is capable of detecting a helicity dependent spin photocurrent;
a fifth electrode and a sixth electrode respectively connected to the spin detector and the substrate, wherein the fifth electrode and the sixth electrode are configured to apply a voltage between the spin detector and the substrate to drive the spin-polarized carriers created in the second multi-layer semiconductor structure to the spin detector; and
a current meter connected to the two electrodes and configured to detect the helicity dependent spin photocurrent flowing through the spin detector.

20. The vertical-type spin-based photodiode according to claim 19, wherein the spin detector is in a form of a bar-shaped channel, the vertical-type spin-based photodiode further comprising:
a seventh electrode and an eighth electrode respectively connected to two opposite ends of the bar-shaped channel of the spin detector to apply a pulsed current into the bar-shaped channel of the spin detector, so as to switch the magnetization direction of the spin detector.
